(19) United States
(12) Patent Application Publication
(10) Pub. No.: US 2023/0042272 A1
(43) Pub. Date: Feb. 9, 2023

(54) Artificial Intelligence Healthcare Assistant Device, System and Method

(71) Applicant: Katharina Mahadeva Cadwell, Reno, NV (US)

(72) Inventor: Katharina Mahadeva Cadwell, Reno, NV (US)

(21) Appl. No.: 17/855,938

(22) Filed: Jul. 1, 2022

Related U.S. Application Data
(60) Provisional application No. 63/230,390, filed on Aug. 6, 2021.

(51) Int. Cl. G16H 10/60; G06Q 40/08; G16H 50/20

(52) U.S. Cl. CPC G16H 10/60; G06Q 40/08; G16H 50/20

(57) ABSTRACT

The present invention relates to an artificial intelligence healthcare assistant device and an associated system allowing patients to better understand their healthcare and receive medical care immediately and periodically using artificial intelligence-based systems. The artificial intelligence device receives medical information of a patient and provides healthcare information, medication, and other treatment options in an easy-to-understand format. The system includes an AI server that also provides analysis on the patient information for detailed healthcare information. The device allows patients to post query to a medical expert and also determines if there is an emergency with the patient. A patient can interact in a variety of ways with the device and reduces chances of confusion in determining a healthcare plan for the patient.

### CROSS-REFERENCE TO RELATED APPLICATION

The present application claims priority to, and the benefit of, U.S. Provisional Application No. 63/230,390, which was filed on Aug. 6, 2021, and is incorporated herein by reference in its entirety.

### FIELD OF THE INVENTION

The present invention relates generally to the field of healthcare personal care devices and healthcare data processing systems. More specifically, the present invention relates to an artificial intelligence driven healthcare device and associated system for providing an interface for patients, their family members, healthcare providers, emergency personnel, and others, to allow them to view, monitor and hear health related information in an easy-to-understand manner. The healthcare device of the present invention leverages artificial intelligence (AI) and allows the patients and others to choose a desired format such as audio, video, graphical, or textual for receiving health related information on the interface and also enables the patients and others to record their queries for a medical provider and to maintain records in order to provide for the wellbeing of the person or persons being monitored. The device of the present invention ensures that the queries of the patients are properly conveyed to healthcare and other care providers and that such issues are resolved properly. Accordingly, the present disclosure makes specific reference thereto. Nonetheless, it is to be appreciated that aspects of the present invention are also equally applicable to other like applications, devices, and methods of manufacture.

### BACKGROUND

By way of background, the healthcare sector is changing quickly and there has been rapid growth in the use of technology to fulfil the needs and requirements of various patients and other caregivers. A lot of development has been done to provide the latest infrastructure and medical devices to the patients, so that the patients are treated quickly in an affordable manner. However, even great strides have been made to provide quick medical treatment, there can be a wide gap in available service offerings related to medical information for hospitalized patients. The patients and their family members may not be able to effectively communicate with the healthcare providers and may fail to get proper medical treatment in a timely manner. In addition, prolonged medical treatment can be expensive and may not be affordable by large segments of the population. This can result in a waste time of both patients and the healthcare workers as well.

Further, patients may not fully understand what is wrong with them while they are suffering from several different ailments, or they may simply discount that something is wrong with them. Assumptions and misconceptions on patients and healthcare providers part regarding medical information can occur with lack of easy-to-understand explanations. Care delivery will then ultimately not be aligned with the patient's values and priorities.

Furthermore, healthcare decisions are not as well informed as they could be, potentially leading to more expensive care. Due to ineffective communication, patients and healthcare providers may get annoyed and become quickly frustrated. People may desire to have a means to enable them to easily communicate with each other, while reducing any confusion between patients and healthcare providers.

Patients are typically required to visit hospitals and doctors for diagnosis. Further, on video calls and phone calls, limited information is accessed by a doctor or other healthcare provider which can lead to ineffective healthcare services and incorrect diagnosis. People desire a system and method that allows them to share information and other concerns with a medical expert and the medical expert can readily obtain historical medical information of the patient.

Therefore, there exists a long-felt need in the art for a device that enables the patients, their family members as well as the healthcare providers to effectively communicate with each other. There is also a long-felt need in the art for a device that ensures that patients are able to properly express their concerns in an effort to seek support and treatment from healthcare workers. Additionally, there is a long-felt need in the art for a medical information device that ensures that the instructions and other health related information are easily and securely conveyed to the patients, their family members, and other authorized recipients. Moreover, there is a long-felt need in the art for a medical device that provides an interface for patients to understand the different ailments and their treatment. Further, there is a long-felt need in the art for a medical information display device that helps to reduce confusion between patients and healthcare providers due to ineffective communication between them. Furthermore, there is a long-felt need in the art for a medical information transfer interface that provides health information in an easy-to-understand manner and reduces the gap or delay in services related to medical information for hospitalized patients. Finally, there is a long-felt need in the art for a medical device that ensures effective communication and quick medical treatment for the patients; thereby saving time and money of the patients as well as the supporting healthcare and insurance systems.

The subject matter disclosed and claimed herein, in one embodiment thereof, comprises an artificial intelligence (AI)-based healthcare system for providing patients with healthcare information and related medical care details in an easy-to-understand format. The healthcare system can be used by hospitals, hospice care, doctors' offices, and other service providers. The system further includes at least one portable AI personal medical device or sensor which functions as a source for medical information of at least one patient. At least one AI server is provided for processing the medical information. The medical information is received from the AI personal medical device at a monitoring location connected to the server. The AI personal medical device performs a number of functions that include receiving personal and medical information of a patient, determining healthcare information of the patient using at least one integrated AI module and comparing that with the received personal and medical information. The healthcare information is displayed on a screen or other readable format of the device. Personal and medical information that is received is uploaded to the AI server and additional healthcare information is obtained from the AI server for displaying on screen or other readout of the device.

In this manner, the system and personal medical device of the present invention accomplishes all of the foregoing objectives and provides users with an artificial intelligence system that is designed to inform them of their medical care in an easy-to-understand format. The device also enables patients and their family to record questions for a medical provider that can be answered by the healthcare provider or other system monitor. The system securely keeps all patient information stored and recorded in a less complex manner and helps in providing accurate healthcare information.

### SUMMARY OF THE INVENTION

The following presents a simplified summary in order to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises an artificial intelligence (AI)-based personal medical interface device, system and method. The personal medical interface device further includes a touch display for displaying a plurality of interfaces to a user and for receiving one or more inputs from a patient or other individual. A microphone is provided for receiving a voice message from a user, a speaker for playing out a voice response message, and a communication interface for establishing a communication channel with one or more external entities. A camera is provided for recording a video of the user, a scanner is used for scanning physical copies of documents, such as medical and insurance records. A personal information module is used for recording personal and medical information of the patient, and an AI module is enabled for running fuzzy algorithms on the data stored in the personal information module for automatically providing health information to the patient. The device is further configured to upload scanned medical records and personal and medical information of the individual to an AI server for machine learning and providing accurate health information to the user or patient.

In yet another embodiment, the medical device is adapted to determine if the individual for whom the medical device is configured is in a medical emergency and further contacts an emergency service and/or key contacts of the individual when an emergency situation is determined to exist.

In yet another embodiment, the emergency is determined based on information received by the medical device from the user.

In yet another embodiment, the medical device provides healthcare information in a plurality of ways including audio, textual, graphical, and other visual representation.

In yet another embodiment, the medical device provides a user interface for allowing the patient to read, write, edit, modify, add to, update, delete, insert, and/or upload medical information and personal medical data.

In yet another embodiment, the user interface is adapted to access the personal medical and other data and the interface is operative on the display on the device.

In yet another embodiment, the medical device can be activated manually or by using a wake-up word or passcode.

In yet another embodiment, an artificial intelligence (AI)-based healthcare system is disclosed. The healthcare system includes at least one portable AI personal medical device functioning as a source for medical information for at least one patient. At least one AI server is provided for processing medical information such that the medical information is received from the AI personal medical device. The AI personal medical device performs a number of functions including receiving personal and medical information of a patient, determining healthcare information of the patient using an integrated AI module and received personal and medical information, and displaying the healthcare information on a screen or other configured element of the device. Personal and medical information that is received is then uploaded to the AI server and more detailed healthcare information is generated from the AI server for displaying on the screen or other configured display of the device.

In yet another embodiment, the AI server has a medical guideline data module for storing medical guideline criteria, a patient information database for storing personal and medical information of one or more patients, and a healthcare information database for storing details of medical experts. A learning and prediction module is provided and analyses the stored medical guideline data using fuzzy rules for providing detailed healthcare information to the patient.

In yet another embodiment, a method for providing healthcare information to a patient in an easy-to-understand format is described. The method includes the steps of initially receiving, by a personal medical device, personal medical data of a patient, and then determining, by the personal medical device, the healthcare status of the patient. An interface of the personal medical device displays the healthcare information of the patient and transmits, by the personal medical device, to a server a request or query from the patient. The server provides a group of medical experts that have a practice set relating to the particular medical request, and a medical expert is then selected to review and provide a response to the query or request. A response is then received from the medical expert and is provided to the individual having placed the original request.

In yet another embodiment, the method further comprising analyzing, by the server, personal medical data and other information of the patient and comparing the received data with stored medical guidelines by applying a plurality of fuzzy rules for deriving healthcare information, recommendations, and treatment for the patient.

In yet another embodiment, the server is an AI server and is a cloud-based server.

Numerous benefits and advantages of this invention will become apparent to those skilled in the art to which it pertains upon reading and understanding of the following detailed specification.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a perspective view of one potential embodiment of the artificial intelligence driven personalized healthcare system of the present invention in accordance with the disclosed architecture;

FIG. 2 illustrates a block diagram showing functional components of the personal AI medical device of the present invention in accordance with the disclosed architecture;

FIG. 3 illustrates a flow diagram depicting a process of providing healthcare information to a patient using the personal AI medical device and the AI healthcare system of the present invention in accordance with the disclosed architecture;

FIG. 4 illustrates a flow chart depicting the process of a patient contacting a medical expert with a request and receiving a response using the personal AI medical device of the present invention in accordance with the disclosed architecture;

FIG. 5 illustrates an exemplary interface displayed by the personal AI medical device to a patient for performing a plurality of functions in accordance with the disclosed architecture;

FIG. 6 illustrates another exemplary interface displayed by the personal AI medical device to a patient displaying health information in accordance with the disclosed architecture; and

FIG. 7 illustrates yet another exemplary interface displayed by personal AI medical device to a medical expert displaying query and information of a patient in accordance with the disclosed architecture.

### DETAILED DESCRIPTION OF THE PRESENT INVENTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there is a long-felt need in the art for a device that enables the patients, their family members and the healthcare providers to effectively communicate with each other. There is also a long-felt need in the art for a system that ensures that patients are able to properly ask their queries from healthcare workers or other care providers. Additionally, there is a long-felt need in the art for a medical information device that ensures that the instructions and other health related information are easily conveyed to the patients, caregivers, insurance providers, and their family members. Moreover, there is a long-felt need in the art for a medical device that provides an interface for patients to understand different ailments, maladies, and their treatment. Further, there is a long-felt need in the art for a medical information display device that reduces confusion between patients, caregivers, insurance providers, and healthcare providers due to ineffective communication between them. Furthermore, there is a long-felt need in the art for a medical information device providing an interface that provides health information in an easy-to-understand manner and reduces the gap in services related to medical information for hospitalized patients. Finally, there is a long-felt need in the art for a medical device, method, and system that ensures effective communication and quick medical treatment for the patients; thereby saving time and money of the patients.

The present invention, in one exemplary embodiment, is a novel artificial intelligence (AI)-based personal medical interface device and system that can be used by patients, doctors, caretakers and others interested parties. The personal medical interface device comprises a touch screen for displaying a plurality of interfaces to a user and for receiving one or more inputs from the user, such as a patient. A microphone is provided for receiving a voice message from the user, and a speaker is used for playing out a voice response message. A communication interface is provided for establishing a communication channel with one or more external entities. A camera is used for recording a video of the user, and a scanner is available for scanning medical records, insurance information and other documents. A personal information module is used for recording personal and medical information of the user, and an AI module runs fuzzy algorithms on the data stored in personal information module for automatically providing health information to the patient. The device further is configured to upload scanned medical records and personal and medical information of the user to an AI server for machine learning, analyzing, comparing, and providing accurate health information to the user.

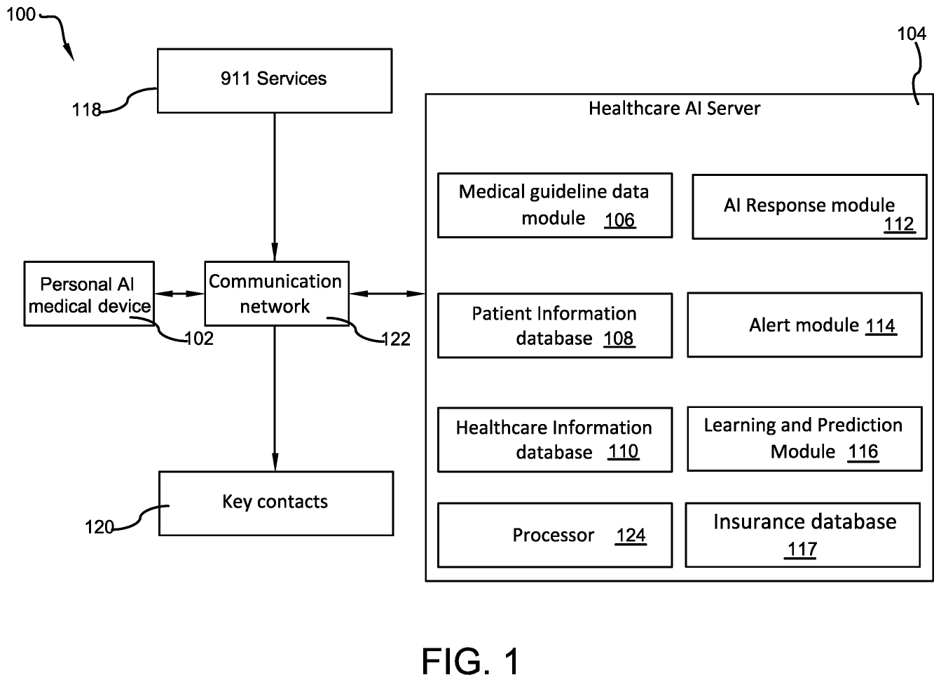

Referring initially to the drawings, FIG. 1 illustrates a perspective view of one potential embodiment of the artificial intelligence driven personalized healthcare system of the present invention in accordance with the disclosed specification. The artificial intelligence driven personalized healthcare system 100 of the present invention is a novel system for providing personalized medical care to patients in easy-to-understand format. The system 100 allows a patient or a user to interact with a medical provider or other healthcare provider without physically going to the medical provider or other location where assistance can be provided. The system 100 can be used by patients, hospitals, doctors' offices and insurance companies and is designed to provide quick and effective care to patients regardless of geographic boundaries and to ensure that the proper insurance is available. An advantage of the system 100 is the use of artificial intelligence for automatically providing accurate medicines, diagnoses, and other information to patients.

More specifically, the system 100 uses an artificial intelligence (AI)-based personal medical device 102 that is designed to be portable, lightweight which can be placed at the bedside of a patient for quick and easy access or be carried from location to location. The AI medical device 102 can be placed at homes, hospitals, long-term care facilities and other suitable areas where assistance and monitoring may be needed allowing a patient, or any other individual, to interact with the device 102 for receiving immediate care, assistance, prognosis, and insights. The personal medical device 102 is configured to communicate with an AI server 104 for providing accurate treatment, information, and care to a patient. It should be noted that the AI medical device 102 of the system 100 has medical guidelines stored in its memory and has the capability of locally providing the treatment, care, and other information. However, the AI medical device 102 communicates with the healthcare server 104 for transmitting patient information and queries, and receiving additional responses and insights.

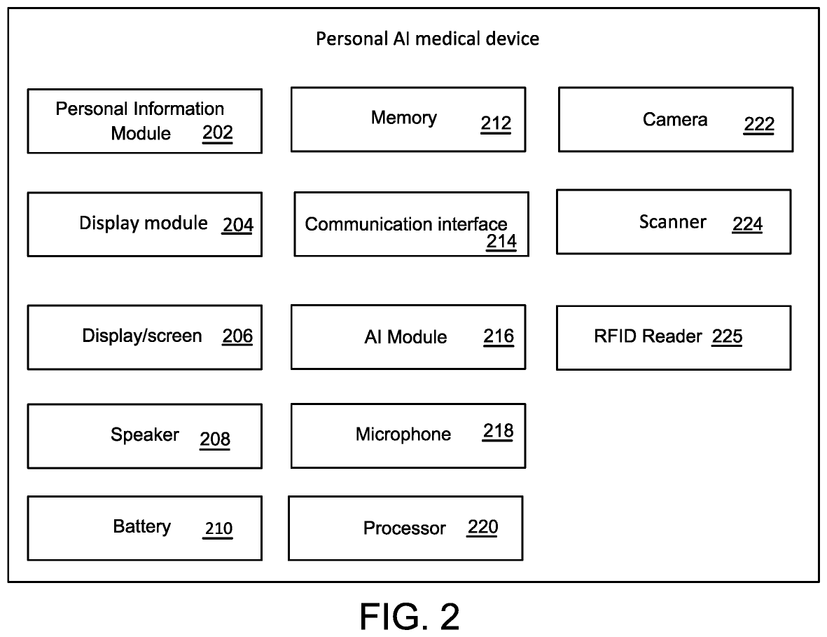

More specifically, the healthcare server 104 has a medical guideline data module 106 that stores medical guidelines used for providing medicines, therapies, treatments, and other information to a patient through the AI personal medical device 102. The medical guideline data module 106 further has recommendations, medications, and other information from medical experts in a variety of fields. Based on condition or symptoms of a patient, recommendations are provided using the medical guideline data module 106. A patient information database 108 securely stores patient information including, but not limited to name, age, location, gender, contact information, medical history, treatment status and other details pertinent to the individual. The patient information database 108 is used for uniquely identifying a patient in the system 100 and accordingly, the patient provided personalized care and treatment. It should be noted that patient information database 108 has a copy of information stored in personal information module (shown in FIG. 2) of the AI medical device 102.

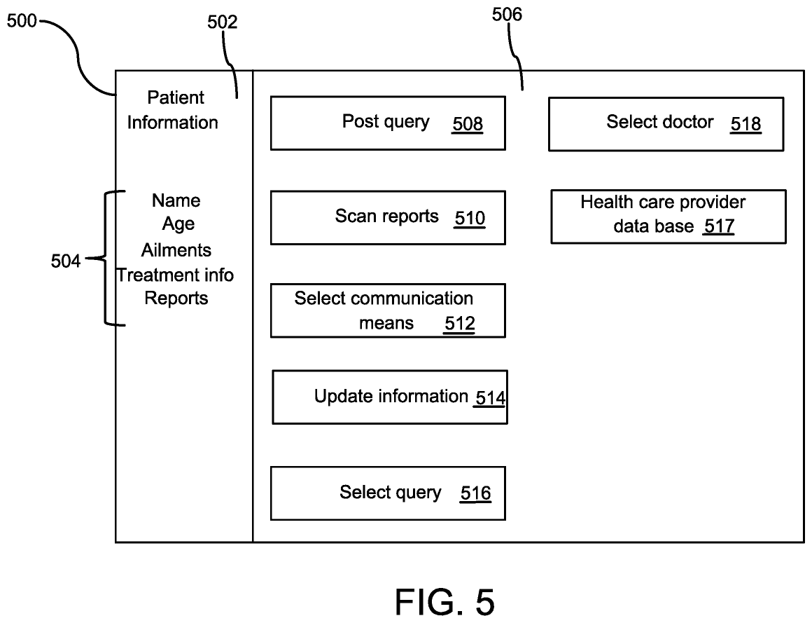

Healthcare information database 110 stores a list and information of medical experts that can be contacted by a patient using the AI personal medical device 102. When a patient, or a family member, records or receives a request or query for a medical expert, the system 100 may choose a relevant medical expert using the healthcare information database 110 to provide a response to the patient. The healthcare AI server 104 has an artificial intelligence (AI) response module 112 that is configured to transmit the response to the medical device 102 in a format selected by a user of the medical device 102. The AI response module 112 is configured to format the response in a format selected by a user (as shown in FIG. 5) so that a response is provided by the medical device 102.

An alert module 114 is configured to generate an alert and may automatically notify a medical expert when the information or a query is received from a patient is determined to be fatal or highly injurious to health. The alert module 114 is triggered by the AI response module 112 and can be used in an SOS situation, for example. The healthcare server 104 uses AI for continuous learning and medical expert advice, recommendations and treatments are continuously fed in the learning and prediction module 116 allowing the system 100 to learn using fuzzy algorithms, compare patterns and other information, and provide accurate care and treatment for the patients. The learning and prediction module 116, along with the medical guideline data module 106, function as the knowledge base of the system 100 that tries to derive answers of user queries, care and treatment for patients.

As stated earlier, the personal AI medical device 102 is configured to locally provide care and treatment to a patient in addition to providing care from the server 104. The AI device 102 is also configured to communicate with a number of different external entities, such as 911 emergency services 118 and key contacts 120 configured by a patient using the system 100. It should be noted that the AI medical device 102 may also contact other entities including, for example: federal agencies, state agencies, diagnostic clinics, medical laboratories, health clinics, physicians, dentists, healthcare workers, urgent care facilities and other relevant administrative entities. All the communication done by the personal AI medical device 102 is in compliance with the Health Insurance Portability and Accountability Act of 1996 (“HIPAA”) and other regulations covering the transfer of medical information of a patient in any country where the device 102 is operated. All the communication done by the personal AI medical device 102 is done through a communication network 122. The communication network 122 may include any of satellite, radio, digital broadband, ultra-wideband (UWB), cable television (CATV), wired communications networks, wireless communications networks, direct broadcast satellite television, multichannel multipoint distribution service (MMDS), wired networks, wireless networks, wireless fidelity (WI-FI), IEEE 802.11 WLAN networks, wireless wide area networks, IEEE 802.16 WWAN networks, (WI-MAX), broadband over power line (BOPL), mobile communications voice or data communications networks, cellular networks, analog or digital cellular networks, mobile networks, packet switched networks, voice over Internet Protocol (VoIP) networks, 2G, 3G, 4G, 5G, 6G, other generation networks, using various exemplary access methods including, e.g., FDMA, CDMA, GSM, GPRS, etc. networks and protocols and combinations of the foregoing.

It should be noted that the AI healthcare server 104 of the system 100 may include a processor 124 for executing the computer executable components of the server 104 as described above. The server 104 may include a communication bus (not shown) for providing coupling and components of the server 104.

The system 100 can be defined as an arrangement of information, data, processes, and information technology that interact to collect, process, store, and provide informational output to support delivery of healthcare information to one or more patients. Information technology includes computer technology (e.g., hardware and software) along with data and telecommunications technology. The system 100 may also include an insurance database 117 which allows healthcare providers, administrators, patients, and family members to check to make sure insurance coverage supports the treatment and other options prescribed by the system.

FIG. 2 illustrates a block diagram showing exemplary functional components of the personal AI medical device 102 of the present invention in accordance with the disclosed specification. The personal AI medical device 102 is a portable device that provides an artificial intelligence driven healthcare interface used by patients, medical experts and other care providers or interested persons. The device 102 has AI capabilities for understanding ailments, maladies, and conditions of a patient and providing health information, treatment, recommendations and more in an easy-to-understand format.

The AI medical device 102 includes a personal information module (PIM) 202 that stores personal and medical information of a patient whose profile is configured in the device 102. The PIM 202 stores personal information such as name, age, contact information, medical history and more. The personal information may be stored in PIM 202 during configuration or set up of the system 102 for a patient. The AI medical device 102 provides an AI user interface to users for operating the device or system 102. The AI user interface is provided on a display or screen using the display module 204. The display module 204 is configured to provide user interface on display of the device 102 allowing a patient to operate the device 102. A display 206 can be a touch display configured to display user interface provided by the display module 204. The display 206 is scratch resistant, break resistant, may be treated with anti-microbial or anti-viral solutions to prevent the spread of germs, bacteria, viruses, or pathogens and has an LED display that requires less power and thus reduces the battery usage of the device 102.

The bedside personal medical device 102 has a speaker 208 for broadcasting health information, recommendations, and other information. The speaker 208 provides convenience to a patient to listen to the health information when the device 102 is not near to the patient so that the patient can read a screen or other informational output. The bedside medical device 102 has a removably attached battery 210 for providing power to the device 102. The battery 210 can be removed and replaced easily and further, the battery 210 can be rechargeable or disposable.

Memory 212 of the device 102 can be a read only memory (ROM), random access memory (RAM) or any other memory known in the art and may constitute an illustrative memory device and/or a non-transitory processor-readable storage medium. The memory 212 may include one or more programming instructions thereon that, when executed by the processor 220, cause the processor 220 to complete various processes, such as the processes described in the present disclosure, e.g., comparing, analyzing, transmitting, receiving, etc. The memory 212 is a computer readable storage media that includes, but is not limited to, random access memory, read only memory, electrically erasable programmable read only memory, flash memory or other memory technology, compact disc read only memory, digital versatile disks or other optical storage, magnetic cassettes, magnetic tape, magnetic disk storage or other magnetic storage devices, or any other medium that can be used to store the desired information and that can be accessed by the processor 220.

A communication interface 214 of the device 102 allows the device 102 to communicate with one or more external entities including the AI server 104 and more as described in FIG. 1. The communication interface 214 is configured to be compatible with all wired and wireless communication means and therefore enables the device 102 to communicate using any communication method as described in FIG. 1 or any other known communication method.

An AI module 216 provides the AI capabilities to the personal client medical device 102. The AI module 216 has fuzzy rules that are trained by either the AI healthcare server 104 or locally using the personal information module 202 of the device or system 102. The AI module 216 also uses the information stored in the medical guidelines data module 106 of the server 104 for learning and enabling the personal medical device 102 to provide accurate health information, recommendations, medication, and treatment to a patient. The AI module 216 also provides healthcare providers necessary information that helps them to remove assumptions and misconceptions about a patient's information or situation. According to the exemplary embodiment, the AI module 216 can be used as a means of quick decisions or actions to be taken in case of emergency, as well as a source of personal health information presented in an easy to read and understand way. The AI module can also be used to alert a hospital or other emergency facility of the present condition of the patient so that the facility is prepared when the individual arrives at the destination. The AI module 216 instructs the processor to contact emergency services, key contacts and more in case of an emergency.

A microphone 218 is disposed in the device 102 allowing a patient or any other user to record a query or make a request. The query may be recorded for a medical expert. Further, the microphone 218 may also be used for recording information for storage in the memory 212 of the device 102. The microphone 218 can be activated by a touch on the display screen or monitor 206 or alternatively using a wake word or passcode.

For receiving accurate information from a patient or any other user, the artificial intelligence healthcare assistant device 102 features a camera 222 that can be used by a user for recording a video. The camera 222 is especially useful in cases where an injury or a physical symptom is to be uploaded to the device 102. The device 102 has an integrated scanner 224 that allows a patient to scan reports and other information easily for a medical expert to view and provide a response. The reports uploaded by a user can also be used for monitoring by the AI module 216 and (also by the server 104) for providing accurate health information to the patients and other users. The system or device 102 may also include a RFID reader 225 which can be used to read RFID tags that may be on a patient's ID bracelet, medical provider's documents or other items. The RFID reader can be used to provide a level of security to only allow authorized individuals to access the system and receive information.

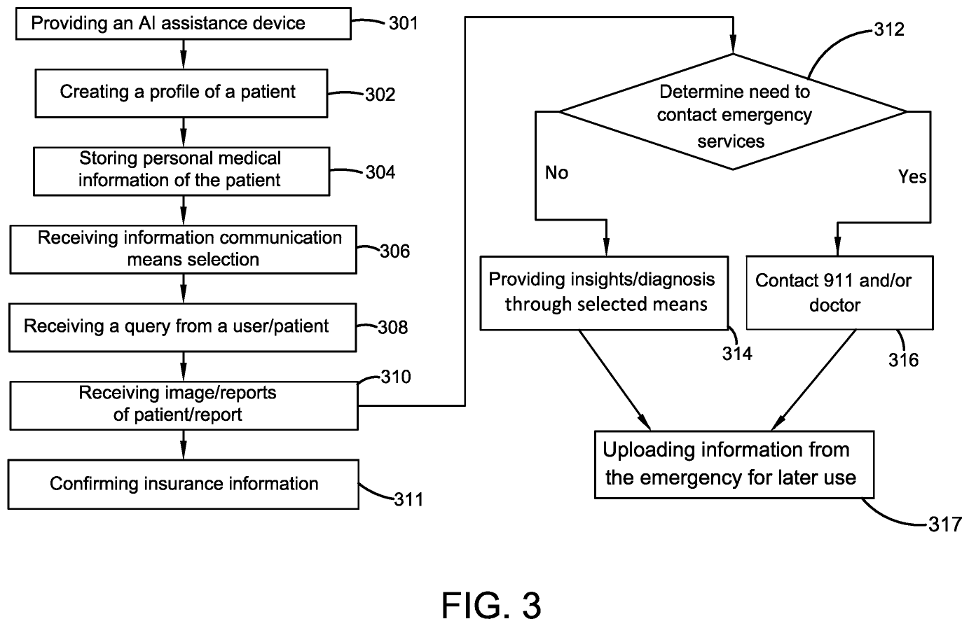

FIG. 3 illustrates a flow diagram depicting a process of providing healthcare information to a patient using the personal AI medical device 102 and the AI healthcare system 100 of the present invention in accordance with the disclosed specification. As illustrated, initially, an AI healthcare assistance device is provided at step 301 and then a profile is created on the personal medical device (Step 302). The profile can be created using the touch display, RFID reader, and/or microphone of the device. After creating a profile, personal medical information may be stored in the local memory of the personal medical device and the medical information can be replicated or otherwise transmitted or transferred to the AI server of the system for machine learning (Step 304). Then, a selection of information means is received from the user (Step 306). The selection enables a user to select a specific way in which information is provided by the personal medical device to the patient/user. A user may select one of audio, visual, and text formats for receiving and conveying information.

Then in step 308, a query may be received from a user or patient wherein the query can be for a medical expert or, alternatively can be for AI module of the system. In the next step 310, images, videos and/or scanned reports are received by the medical device for inspection. An insurance module may be accessed at step 311 in order to determine coverage amounts and benefits. Thereafter, in step 312, the information of the patient is monitored and processed by the system 100 to determine if there is an emergency situation with the patient. If it is determined that there is no emergency, then the process moves to step 314 and healthcare information is provided to the patient through the personal medical device. If it is determined that there is an emergency, then the process moves to step 316 and the personal medical device may contact one or more of 911-type emergency service, a doctor or a key contact of the patient. After the emergency is resolved or treatment prescribed, the information from the event is uploaded to the system at step 317.

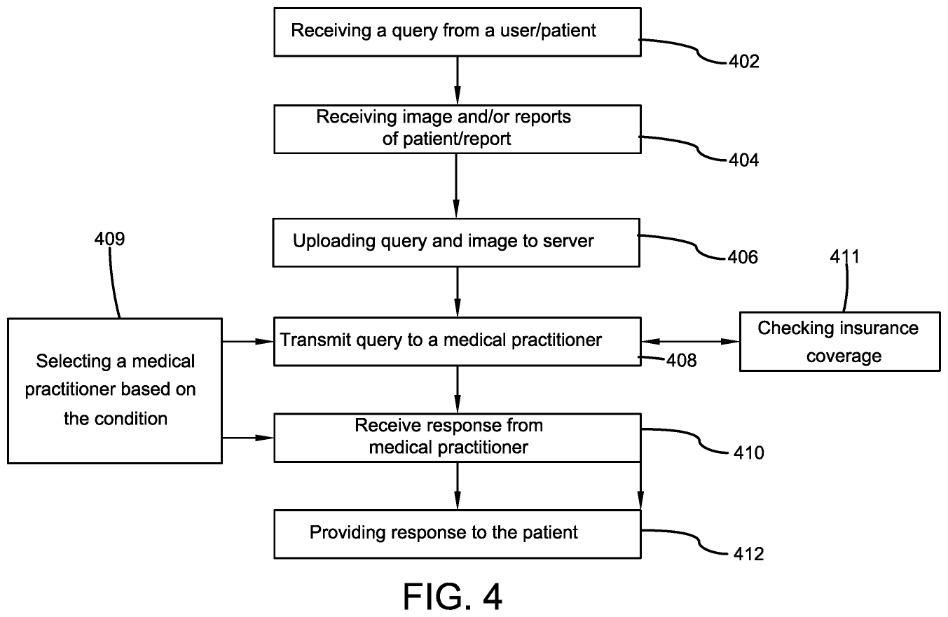

FIG. 4 illustrates a flow chart depicting a process of a patient contacting a medical expert for a query and receiving response using the personal AI medical device of the present invention in accordance with the disclosed specification. As illustrated, initially a query is received by the personal medical device from a patient (Step 402). The patient/user can submit a query through a voice note using microphone, video monitor, or using the touch display of the device. Then, the personal medical device may receive one or more images and reports that can be viewed by a medical expert (Step 404). Then, the patient's query and other information including images and reports are uploaded to the AI server of the system of the present invention (Step 406). Based on analysis of the query, a relevant medical expert is selected from the healthcare information database for responding to query of the patient (Step 408). It should be noted that AI module of the personal medical device can also provide an immediate response based on the learning module of the system.

Based on selection of the medical expert, at step 409, a response is received from the medical expert by the AI medical device (Step 410). Finally, using the selected method of receiving information by the patient, the AI medical device provides the health information and other recommendations to the patient (Step 412).

It should be noted that authorized medical experts and practitioners can use the system 100 to guide, review, and monitor patient care for compliance with standards of care and patient care management in addition to checking insurance coverage at step 411 available to the patient and tailoring the treatment to be in line with such coverage. Further, the AI medical device 102 can analyze medical data for diagnostic purposes as well as to identify potential health issues, suggest treatment options, follow-up testing, and provide other information in support of a patient's health care providers. The AI medical device 102 may use raw clinical data, scanned reports, current clinical practices using a patient's advanced directive, demographics, psychological, currently accepted medical guidelines, prior physician practices, and biological data, for its decision making.

The AI medical device 102 also record observations, notes, or any other health related issues of a patient for further use, according to an exemplary embodiment. The AI medical device 102 may be also used to record new doctor/patient observations.

FIG. 5 illustrates an exemplary interface displayed by personal AI medical device 102 to a patient for performing a plurality of functions in accordance with the disclosed specification. The AI medical device 102 is configured to provide an interface 500 that is used by a patient or a user for interacting with the device 102. The interface 500 has a patient information section 502 that may include name, age, ailments, maladies, treatment information and other relevant information (collectively referred by numeral 504) of the patient. It should be noted that the patient information section 502 can be configured to be hidden or selectively displayed for maintaining privacy of the patient. The full display of information can be triggered by use of a password, passcode, bar code scanning or RFID tag scanning.

A main interface section 506 is configured to display a plurality of controls that allow a patient to interact with the artificial intelligence healthcare assistant device 102 and the system 100. A post query control 508 allows a patient to submit a query for a medical expert. The query can be submitted using the display or through a voice message using the microphone of the device 102. A scan reports control 510 allows a user to scan their reports using an integrated scanner of the device 102 and thereby allows the device 102 and the AI server 104 to store the scanned reports for analysis.

A communication means selection control 512 allows a patient to select a manner for receiving information in an easy-to-understand format. The device 102 can provide information to a patient using one of audio, visual, and text formats or any combination of the formats can be selected by a user/patient based on the preference. An update information control 514 allows a patient to update personal and medical information allowing the system 100 to store the updated information for providing tailored and customized care to the patient.

An advantage of the medical device 102 is that the device 102 provides AI built queries pertaining to ailments or other maladies. The queries help a patient to frame a specific query and thereby receive an accurate health information. The queries can be displayed and selected by a patient using the select query control 516.

A patient may select a treating physician or other care professional selecting a specific doctor/medical expert, and the device 102 provides a select doctor control 518 module which accesses a database 517 that allows a user to post requests or queries to a specific doctor or other provider. Alternatively, the AI system automatically selects the best doctor that matches requirements and the profile of the user/patient to provide a response to the query of the patient/user by accessing the database 517.

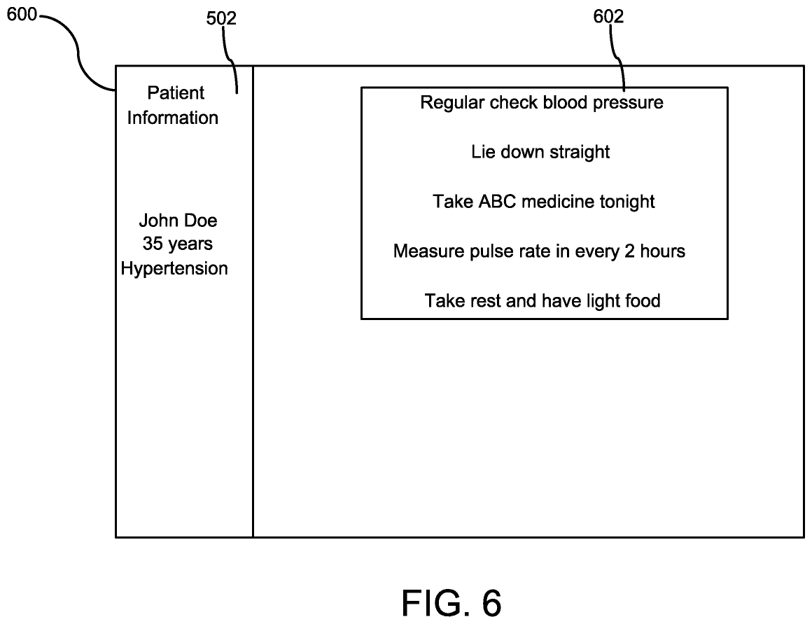

FIG. 6 illustrates another exemplary interface displayed by personal AI medical device 102 to a patient displaying health information in accordance with the disclosed specification. As illustrated, the user interface 600 displays the patient information portion 502 and health and medical care section 602 for review of a patient/user. The health and medical care section 602 includes one or more healthcare information, care, recommendation, medication, and any other information useful for the patient. All the information is customized for the patient and reduces the chance of any confusion occurring due to how healthcare treatments will be planned by medical experts.

The artificial intelligence healthcare assistant device 102 provides the patient with the information that is needed for understanding of their medical condition(s) and through this, the individual can assist in informed decision making regarding a patient's health. The AI module of the device 102 and the server 104 provides predictive healthcare assessment to the patients.

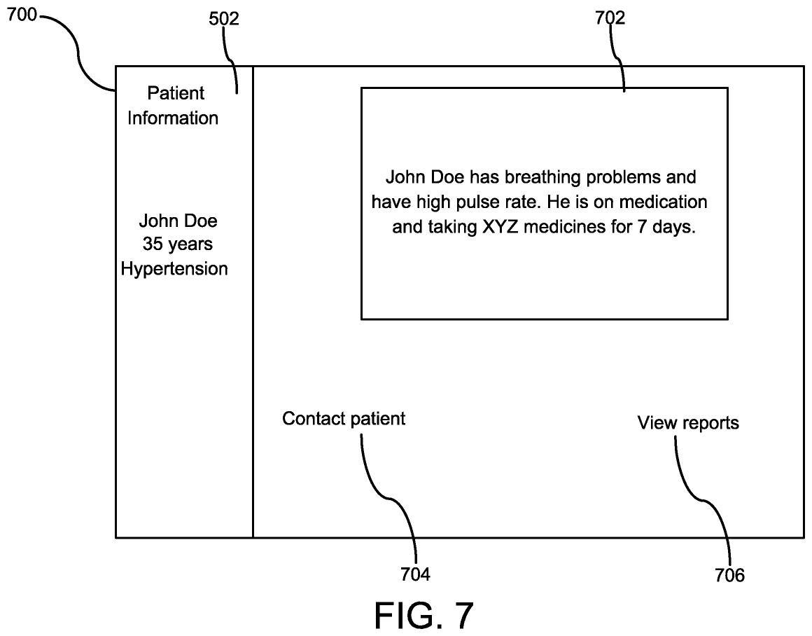

FIG. 7 illustrates yet another exemplary interface displayed by personal AI medical device 102 to a medical expert displaying a query and information of a patient in accordance with the disclosed specification. As illustrated on the interface 700, the patient information portion 502 displays personal and medical information of a patient. The personal and medical information transmitted and stored in the system 100 of the invention is in accordance with the regulations provided by regulatory bodies of the countries in which the device 102 and the system 100 is used.

A query portion 702 displays the query of a user/patient along with any additional information predicted or learned by the artificial intelligence of the device 102. The query portion 702 allows the doctor to prescribe a treatment along with any additional recommendation and information. The interface 700 further provides a contact patient control 704 to allow the doctor to contact the patient, and a view reports control 706 to allow the doctor or other care provider to view reports of the user/patient.

The display interfaces in the present disclosure are provided as just one example of the many possible embodiments of such user interfaces and embodiments. Minor changes to such user interfaces are intended to be within the scope of this disclosure. As one example, the present disclosure makes reference to various controls, such as buttons, fields, selectable controls, check boxes, tabs, menus, and the like. Other embodiments can be made by selecting alternative web page displays.

It should be noted that the artificial intelligence healthcare assistant device 102 and the AI server 104 use data that has been previously stored as a portion of a patient's medical records for learning and providing accurate health information to the patient. Examples of patient medical information includes, but is not limited to, previous health history, previous surgical procedures, laboratory results, current and past medications taken, family history, and/or the like.

Also, medical journals, data from guidelines, and/or the like are also used for providing treatment and health information to a patient. All the data may be updated in real-time as new information is received to ensure that the recommendations provided to a patient, as described herein, reflects the most accurate and up-to-date information.

Further, training modules used by AI module of the device 102 and the learning and prediction module of the server 104 may be automatically rerun, periodically, occasionally, or with each individual patient request, for an individual prediction. That is, the algorithms may be automatically recalibrated, re-evaluated, and reselected periodically, occasionally, or otherwise, using the then current data. More data is more statistically reliable, so automatically re-running of the training module with more data makes the model predictions increasingly better, that is this machine learning is improving the prediction results. This provides an automatic artificial intelligence pipeline of ever-improving predictive models.

Certain terms are used throughout the following description and claims to refer to particular features or components. As one skilled in the art will appreciate, different persons may refer to the same feature or component by different names. This document does not intend to distinguish between components or features that differ in name but not structure or function. As used herein “personal AI medical device”, “AI medical device”, “personal medical device”, and “medical device”, are interchangeable and refer to the personal AI medical device 102 of the present invention. Similarly, as used herein “artificial intelligence driven personalized healthcare system”, “AI healthcare system”, and “system” are interchangeable and refer to the artificial intelligence driven personalized healthcare system 100 of the present invention.

Notwithstanding the foregoing, the AI driven personalized healthcare system 100 and the personal AI medical device 102 of the present invention can be of any suitable size and configuration as is known in the art without affecting the overall concept of the invention, provided that it accomplishes the above stated objectives. One of ordinary skill in the art will appreciate that the AI driven personalized healthcare system 100 and the personal AI medical device 102 as shown in the FIGS. are for illustrative purposes only, and that many other sizes and shapes of the AI driven personalized healthcare system 100 and the personal AI medical device 102 are well within the scope of the present disclosure. Although the dimensions of the AI driven personalized healthcare system 100 and the personal AI medical device 102 are important design parameters for user convenience, the AI driven personalized healthcare system 100 and the personal AI medical device 102 may be of any configuration that ensures optimal performance during use and/or that suits the user's needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A personal healthcare system comprising:
a personal healthcare device having a communication module comprising an information relating to an emergency and other contact information for a patient using the personal healthcare system;
a server connected to the personal healthcare device by the communication module, the server having a plurality of databases; and
a controller for accessing one or more of the plurality of databases, wherein the communication module is used for submitting a request or query to the server to obtain a medical assistance or treatment for the patient.

2. The personal healthcare system as recited in claim 1, wherein the plurality of databases comprises a patient information database and a healthcare information database.

3. The personal healthcare system as recited in claim 1 further comprising a response module for transmitting a response to the personal healthcare device.

4. The personal healthcare system as recited in claim 1 further comprising a user alert module configured to generate an alert.

5. The personal healthcare system as recited in claim 4, wherein the user alert module can be used to generate an emergency alert.

6. The personal healthcare system as recited in claim 1 further comprising a learning and predictive module that utilizes a fuzzy algorithm.

7. The personal healthcare system as recited in claim 2, wherein the plurality of databases further comprises an insurance database.

8. The personal healthcare system as recited in claim 7, wherein the insurance database is used to determine an insurance coverage for a treatment.

9. The personal healthcare system as recited in claim 2, wherein the patient information module comprises information relating to a name, an age, a location, a gender, a contact information, a medical history, and a treatment status pertinent to the patient.

10. The personal healthcare system as recited in claim 3, wherein the response module configures a response to be transmitted in a format requested by the patient.

11. The personal healthcare system as recited in claim 10, wherein the format is one of a video, an audio, a text or a graphic.

12. The personal healthcare system as recited in claim 1, wherein the communication module uses a network selected from a group consisting of a satellite, a radio, a digital broadband, an ultra-wideband (UWB), a cable television (CATV), a wired communications network, a wireless communications network, a direct broadcast satellite television, a multichannel multipoint distribution service (MMDS), a wired network, a wireless network, a wireless fidelity (WI-FI), an IEEE 802.11 WLAN network, a wireless wide area networks, an IEEE 802.16 WWAN network, a WI-MAX, a broadband over power line (BOPL), a mobile communications voice or data communications network, a cellular network, an analog or digital cellular network, a mobile network, a packet switched network, a voice over Internet Protocol (VoIP) network, a 2G network, a 3G network, a 4G network, a 5G network and a 6G network.

13. A method of using a personal healthcare system, the method comprising the steps of:
providing an artificial intelligence device;
creating a profile of a patient;
storing a medical information of the patient;
receiving information concerning a communication format from a communication module connected to a communication network;
receiving a request from the patient for a healthcare service; and
determining whether an emergency exists based on the request.

14. The method of using a personal healthcare system as recited in claim 13 further comprising a step of confirming an insurance information after the step of receiving a request from the patient.

15. The method of using a personal healthcare system as recited in claim 13 further comprising a step of contacting an emergency service if an emergency exists after the step of determining whether an emergency exist.

16. The method of using a personal healthcare system as recited in claim 13 further comprising a step of providing a medical diagnosis if no emergency exists after the step of determining whether an emergency exist.

17. The method of using a personal healthcare system as recited in claim 16 further comprising a step of updating information from the emergency determination after the step of determining whether an emergency exists.

18. A method for seeking a treatment using a personal healthcare system, the method comprising the steps of:
receiving a query or request from a patient;
receiving one of an image or report from the patient;
uploading the image or report and the query or request to a server;
transmitting the image or report and the query or request to a medical provider;
receiving a response from the medical provider; and
providing the response to the patient.

19. The method for seeking treatment using a personal healthcare system as recited in claim 18 further comprising a step of selecting the medical provider based on the request or query after the step of transmitting.

20. The method for seeking treatment using a personal healthcare system as recited in claim 18 further comprising a step of checking an insurance coverage for the patient after the step of transmitting.
